(19) United States
(12) Patent Application Publication
(10) Pub. No.: US 2011/0009856 A1
(43) Pub. Date: Jan. 13, 2011

(54) Combination Radio Frequency Device for Electrosurgery

(76) Inventors: Glen Jorgensen, Jacksonville, FL (US); Steve Woolfson, Boston, MA (US); William G. Dennis, Jacksonville, FL (US)

Correspondence Address: HUNTON & WILLIAMS LLP;INTELLECTUAL PROPERTY DEPARTMENT, RIVERFRONT PLAZA, EAST TOWER, 951 EAST BYRD ST., RICHMOND, VA, 23219-4074, US

(21) Appl. No.: 12/832,422

(22) Filed: Jul. 8, 2010

Related U.S. Application Data
(60) Provisional application No. 61/223,886, filed on Jul. 8, 2009.

(51) Int. Cl. A61B 18/18

(52) U.S. Cl. 606/33

(57) ABSTRACT

A radio frequency (RF) device for use in electrosurgery is provided. The RF device comprises a flexible support member having an actuator passage extending therethrough and a flexible actuator member at least partially disposed within the actuator passage. An insulator housing is connected on its proximal side to the distal end of the actuator so that when the flexible actuator member is rotated, the insulator housing rotates about a rotation axis through the distal actuator end. The device also comprises at least one electrode extending distally from the insulator housing, the at least one electrode having first and second energy transmitting surfaces configured so that if the first energy transmitting surface is positioned to deliver electrical current with the insulator housing in a first angular orientation, rotation of the insulator housing to a second orientation positions the second energy transmitting for transfer of electrical current.

### CROSS-REFERENCE TO RELATED APPLICATIONS

The present application claims the benefit of U.S. provisional application 61/223,886, filed Jul. 8, 2009, which is incorporated herein by reference in its entirety.

### FIELD OF THE INVENTION

The invention generally relates to radio frequency devices for use in electrosurgical operations, and more particularly to an electrosurgical device that is capable of operating in both a cutting mode and a coagulating mode.

### BACKGROUND OF THE INVENTION

Electrosurgery is the application of an electric current to cut or coagulate tissue. Typically, electrosurgical devices use a high-frequency electric current to heat the tissue. Electrosurgery is a very popular procedure for surgical operations because it allows the surgeon to make precise cuts with limited blood loss. Electrosurgical devices are frequently used during surgical operations (e.g., dermatological, gynecological, cardiac, plastic, ocular, spine, ENT, orthopedic, urological, neuro and general surgical procedures) to aid in limiting blood loss.

Electrosurgery is performed using an Electrosurgical Generator (also referred to as a Power Supply or a Waveform Generator) and a hand-piece including one or several electrodes, sometimes referred to as a radio frequency (“RF”) knife or device.

When voltage is applied across a material, it produces an electric field that exerts a force on charged particles. A flow of free charge carriers—electrons and ions—is called an electric current. In metals and semiconductors the charge is carried primarily by electrons, whereas in liquids the charge is carried primarily by ions. Electrical conduction in biological tissues is primarily due to the conductivity of the interstitial fluids, and thus is predominantly ionic. Transition between the electronic and ionic conduction is governed by electrochemical processes at the electrode-electrolyte interface. The value of electric current, I, through a material is determined by the applied voltage, V, and the material's resistance, R, according to Ohm's law: I=V/R.

Electric current of a constant polarity is referred to as Direct Current (DC). A current of alternating polarity is referred to as Alternating Current (AC). Its frequency is measured in cycles/second or Hertz (Hz). Current flowing through a resistor causes the generation of Joule heating. In other words, the resistance of the tissue converts the electric energy of the voltage source into heat (thermal energy) which causes the tissue temperature to rise. The deposited electric power (energy per unit of time) can be calculated using: P=I×V=I²×R=V²/R where P represents the electric power (typically measured in Watts).

In the absence of heat conduction, the rate of temperature rise, dT/dt (also referred to herein as dQ/dt), in a heated object is proportional to the deposited power P, and inversely proportional to its heat capacity. This is in turn proportional to the mass, m, of the object and its specific heat capacity, c. The formula is dT/dt=P/(c×m).

Larger amounts of heat are required to increase the temperature of a heavier object. Therefore, when heat is generated in a small region of an object, the temperature of that localized region will rise much faster than if the same amount of heat is evenly dispersed over the entire object.

Current density, j, is a measure of the concentration of electric current. A higher electric current density results in a higher concentration of Joule heating. Power density, p, generated by the electric current in the material is proportional to the square of the current density, and to the material's resistivity, g. The formula is: p=j²×g.

In the absence of heat conduction, the rate of local temperature rise is proportional to the power density, p, produced in that region of tissue, and inversely proportional to its specific heat capacity and density, p. The formula is: dT/dt=p/(c×p).

Neural and muscle cells are electrically-excitable, meaning that they can be stimulated by an electric current. In human patients such stimulation may cause acute pain, muscle spasms, and even cardiac arrest. Sensitivity of the nerve and muscle cells to an electric field is due to the voltage-gated ion channels present in their cell membranes. Stimulation threshold does not vary much at low frequencies, but the threshold starts increasing with decreasing duration of a pulse (or a cycle) when it drops below a characteristic minimum, which is referred to as “chronaxie”. Typically, chronaxie of neural cells is in the range of 0.1-10 ms, so the sensitivity to electrical stimulation (inverse of the stimulation threshold) decreases with increasing frequency in the kHz range and above. To minimize the effects of muscle and neural stimulation, electrosurgical equipment typically operates in the RF range of 100 kHz to 5 MHz.

Operation at higher frequencies also helps minimize the amount of hydrogen and oxygen generated by electrolysis of water. This is an especially important consideration for applications in liquid medium in closed compartments, where the generation of gas bubbles may interfere with the electrosurgical procedure. For example, bubbles produced during an operation inside an eye may obscure a field of view.

There are several commonly used electrode configurations or circuit topologies, the most common being bi-polar and mono-polar.

In bi-polar configuration, the voltage is applied to the patient using a pair of similarly-sized electrodes. For example, in one electrosurgery bi-polar configuration, special forceps are used where one tine is connected to one pole of the AC generator (i.e., the supply electrode) and the other tine connected to the other pole of the generator (i.e., the return electrode). When a piece of tissue is held by the forceps, a high frequency electric current flows from one tine to the other, heating the intervening tissue.

In bi-polar electrosurgery, both the supply electrode and return electrode functions are performed at the site of surgery. The two tines of the forceps perform both the supply and return electrode functions. Only the tissue grasped is included in the electrical circuit. Because the return function is performed by one tine of the forceps, no patient return electrode is needed.

In mono-polar electrosurgery, the patient typically lies on top of the return electrode, which is often a relatively large metal plate or a flexible metalized plastic pad that is connected to the return electrode of the AC source, and the surgeon uses a pointed probe to make contact with the tissue. The electric current flows from the probe tip through the body to the return electrode and then back to the electrosurgical generator. Since electric current spreads from the pointed electrode as it enters the body, the current density rapidly (quadratically) decreases as it traverses tissue farther into the body. Because the rate of heating is proportional to the square of current density, the heating occurs in a very localized region, i.e., only near the probe tip. Nevertheless, on an extremity such as a finger, for example, there is limited cross-sectional area for the return current to spread across, which might result in higher current density and some heating throughout the volume of the extremity.

Various electrosurgical modalities are available. For example, in the electrosurgical cutting modality, tissue is divided with electric sparks that focus intense heat at the surgical site. The surgeon produces maximum current concentration by sparking the tissue. To create this spark, the surgeon holds the electrode slightly away from the tissue. This will produce the greatest amount of heat over a very short period of time and result in vaporization of tissue.

In the electrosurgical fulguration modality (sparking with the coagulation waveform) tissue is coagulated and charred over a wide area. In this mode the duty cycle (i.e., ratio of ON time to the period) is only about 6%, so less heat is produced. The result is the creation of a coagulum rather than cellular vaporization. The coagulation waveform has significantly higher voltage than the cutting current to overcome the high impedance of air. Use of high voltage coagulation current has implications during minimally invasive surgery.

In the electrosurgical desiccation modality the electrode is in direct contact with the tissue that is exposed to air. Desiccation is achieved most efficiently with the “cutting” current. By touching the tissue with the electrode, the current concentration is reduced, thus less heat is generated and little or no cutting action occurs. The tissue cells dry out and form a coagulum rather than vaporize and explode.

Each of these electrosurgical modalities can use a different electrical waveform. Electrosurgical generators are able to produce a variety of electrical waveforms and frequencies. As waveforms change, so will the corresponding tissue effects. The surgeon is able to vaporize or cut tissue using a continuous single frequency sine wave. This waveform produces heat very rapidly. Energy delivery beyond the vaporization threshold can continue if sufficiently high voltage is applied (>+/−200 V) to ionize vapor and convert it into a conductive plasma. Rapid tissue heating leads to explosive vaporization of interstitial fluid. Electric current continues to flow from the metal electrode through the ionized gas into the tissue. Rapid overheating of tissue results in its vaporization, fragmentation, and ejection of fragments, allowing for tissue cutting. In applications of a continuous wave, the heat diffusion typically leads to the formation of a significant thermal damage zone at the edges of the lesion. Open circuit voltage in electrosurgical waveforms is typically in the range of 300-10,000 V peak-to-peak.

Using an intermittent or pulsed waveform causes the generator to modify the waveform so that the duty cycle is reduced. Duty cycle is defined as the ratio of the ON time to the period (the time of a single ON-OFF cycle). A lower duty cycle produces less heat. The process of altering an amplitude of a periodic waveform is called modulation. The intermittent or pulsed waveform will produce less heat. Instead of tissue vaporization, a coagulum is produced. A “blended current” is not a mixture of both cutting and coagulation current, but rather a modification of the duty cycle.

Using bursts of several tens of microseconds in duration, the tissue can be cut while the size of the heat diffusion zone does not exceed the cellular scale. Heat accumulation during repetitive application of bursts can also be avoided if sufficient delay is provided between the bursts, thus allowing the tissue to cool down. The proportion of ON time to OFF time can be varied to allow control of the heating rate. For coagulation, the average power is typically reduced below the threshold of cutting. Typically, sine waves are turned ON and OFF in a rapid succession. The overall effect is a slower heating process, which causes tissue to coagulate.

For convenience in using prior art electrosurgical devices, surgeons routinely cut with the coagulation current and coagulate with the cutting current. It may be necessary or desirable, however, to adjust power settings and electrode size to achieve a more precise surgical effect.

The rate at which heat is produced is not the only variable that determines whether a waveform vaporizes tissue or produces a coagulum. Any waveform can vaporize tissue or produce a coagulum by modifying the variables that impact tissue effect. In addition to waveform and power setting, the other variables that impact tissue effect include: 1) the size of the electrode; 2) timing; 3) how the electrode is manipulated; 4) the type of tissue; and 5) eschar (scab tissue).

Size of the electrode: The smaller the electrode, the higher the current concentration. Consequently, the same tissue effect can be achieved with a smaller electrode, even if the power setting is reduced.

Time: At any given setting, the longer the generator is activated, the more heat is produced. And the greater the heat, the farther it will travel to adjacent tissue, an effect known as thermal spread.

Manipulation of the electrode: This can determine whether vaporization or coagulation occurs. This is a function of current density and the resultant heat produced and sparking to the tissue versus holding the electrode in direct contact with the tissue.

Type of Tissue: Different tissues vary widely in resistance and the impact of current on tissue effect is proportional to the tissue's resistance.

Eschar: Eschar is scar tissue that forms especially after a burn, and has a relatively high resistance. Keeping electrodes clean and free of eschar enhances performance by maintaining lower resistance within the surgical circuit. As the layer of eschar increases the effective current delivered to the tissue decreases.

Whether cutting or coagulating, the change in performance caused by eschar requires the surgery team to either: 1) increase the voltage, or 2) remove the electrode from the body for cleaning. Both of these procedures have risks. Increasing the voltage always increases the risks in electrosurgery. Higher voltage stresses the safety insulation around the electrode and may cause insulation failure. Higher voltage also increases the likelihood that a secondary grounding path will be included in the circuit. With higher voltage, the current may take an unexpected and unpredictable path through the patient, thus causing damage to the patient without immediately alerting the surgical team of the problem.

The second alternative of removing and cleaning eschar from the electrode often ruins the coating on the electrode. Most electrodes are protected from eschar with either a Teflon® coating or an elastomeric silicone coating. Neither coating prevents eschar build-up, however; yet both coatings facilitate the removal of the eschar.

### SUMMARY OF THE INVENTION

An aspect of the present invention provides a radio frequency (RF) device for use in electrosurgery to deliver electrical current from an electrical source to target tissue of a patient. The RF device comprises a flexible support member having an actuator passage extending therethrough from a proximal support member end to a distal support member end. The RF device further comprises a flexible actuator member at least partially disposed within the actuator passage. The flexible actuator member is selectively rotatable relative to the flexible support member and has a distal actuator end adjacent the distal support member end. The device also comprises an insulator housing having a proximal side and a distal side. The insulator housing is connected on its proximal side to the distal actuator end so that when the flexible actuator member is rotated, the insulator housing rotates about a rotation axis through the distal actuator end. The insulator housing is rotatable between at least first and second angular orientations. The device also comprises at least one electrode in selective communication with the electrical source. The at least one electrode extends distally from the distal side of the insulator housing and has first and second energy transmitting surfaces configured so that if the first energy transmitting surface is positioned to deliver electrical current to the target tissue with the insulator housing in the first angular orientation, rotation of the insulator housing to the second angular orientation positions the second energy transmitting for transfer of electrical current to the target tissue.

Another aspect of the present invention also provides a radio frequency (RF) device for use in electrosurgery to deliver electrical current from an electrical source to target tissue of a patient. The RF device comprises an insulator housing having a housing passage extending therethrough. The housing passage has a proximal passage opening in a proximal side of the insulator housing and a distal passage opening in a distal side of the insulator housing. A first electrode is attached to the distal side of the insulator housing. The first electrode has an external first energy transmitting surface and an internal electrical contact surface exposed to the housing passage. The RF device further comprises a second electrode in selective communication with an electrical energy source. The second electrode has a second energy transmission surface at a distal electrode end and an electrical supply contact. The second electrode is slidably disposed within the housing passage so that it is movable between a first position in which the second electrode is withdrawn within the housing passage and the electrical supply contact is in electrical communication with the internal electrical contact of the first electrode and a second position in which at least a portion of the second electrode extends distally outward from the distal passage opening and the electrical supply contact is not in electrical communication with the internal electrical contact of the first electrode. The RF device also comprises a flexible actuator member having a distal actuator end slidably disposed within the housing passage. The distal actuator end is attached to a proximal end of the second electrode so that axial translation of the flexible actuator member causes corresponding motion of the second electrode relative to the housing passage.

It is to be understood that both the foregoing general description and the following detailed description are exemplary and explanatory only, and are not restrictive of the invention as claimed. The accompanying drawings which constitute a part of the specification, illustrate certain embodiments of the invention and, together with the detailed description, serve to explain the principles of the invention.

### BRIEF DESCRIPTION OF THE DRAWINGS

The invention can be more fully understood by reading the following detailed description together with the accompanying drawings, in which like reference indicators are used to designate like elements, and in which:

FIG. 1 is a sectional view of an RF device according to an embodiment of the invention in a coagulating mode.

FIG. 1A is a cross-sectional view of the RF device of FIG. 1.

FIG. 2 is a sectional view of the RF device of FIG. 1 in a tissue cutting mode.

FIG. 3 is a sectional view of an RF device according to an embodiment of the invention.

FIG. 4 is a sectional view of an RF device according to an embodiment of the invention in a coagulating mode.

FIG. 4A is a cross-sectional view of the RF device of FIG. 4.

FIG. 5 is a sectional view of the RF device of FIG. 4 in a tissue cutting mode.

FIG. 6 is a sectional view of an RF device according to an embodiment of the invention.

FIG. 7 is a plan view of the bi-polar RF device of FIG. 4 with the electrodes in a distal position, and a proximal position shown in phantom.

FIG. 8 is a plan view of an RF device according to an embodiment of the invention.

FIG. 9 is a side view of an RF device according to an embodiment of the invention in a coagulation mode.

FIG. 10 is a side view of the RF device of FIG. 9 in a cutting mode.

FIG. 11 is a perspective view of the RF device of FIG. 9.

FIG. 11A is a front cross-sectional view of the RF device of FIG. 11.

FIG. 12 is a perspective view of an RF device according to an embodiment of the invention.

FIG. 13 is a perspective view of a hub and a sheath device that may be used in conjunction with embodiments of the invention.

FIG. 14 is a perspective view showing the use of an RF device of the invention with the hub and sheath of FIG. 13.

### DETAILED DESCRIPTION OF THE INVENTION

There is a need in the prior art for an RF device that does not require changing out electrodes on the RF device during an electrosurgical operation, and that more effectively limits eschar buildup, and that can deliver more precise results when functioning in both the coagulating and cutting modes. The inventors have developed an RF device that achieves these and other advantages by having an energy transmitting surface for the purpose of focusing the electrical current to cut tissue, and also another energy transmitting surface for the purpose of spreading the electrical current to coagulate tissue, each energy transmitting surface on the same RF device and positionable between multiple spatial and angular orientations. By separating the cutting and coagulating modes in the same RF device, eschar buildup can be reduced, the efficiency of the RF device is increased, and the precision of tissue effect is enhanced. Further precision can be attained by having an RF device tip that is bendable and/or rotatable. In addition to the disclosure below, more information about the bendability and rotatability of the RF device can be found in U.S. patent application Ser. Nos. 11/643,740, 12/119,799, and 12/399,471 which are herein incorporated by reference in their entirety.

The present invention provides for an RF device that more effectively limits eschar buildup, increases efficiency, and delivers better precision when functioning in the various electrosurgical modalities. The RF device disclosed herein achieves these and other advantages by, inter alia, having on the same end of the RF device both an energy transmitting surface for the purpose of focusing the electrical current to cut tissue, and an energy transmitting surface for the purpose of spreading the electrical current to coagulate tissue, among other possible electrosurgical modalities. By separating the cutting and coagulating modalities, for example, in the same RF device, eschar buildup can be reduced, the efficiency of the RF device is increased, and the precision of tissue effect is enhanced.

The present invention provides for further precision of the RF device by utilizing a rotatable-tip that can present a variety of electrode orientations relative to the target tissue. By altering the electrode orientation between, for example, tangential and perpendicular orientations, different electrosurgical modalities can be performed with the same tip of an RF device. The RF device disclosed herein may comprise a flexible support member in the shape of a spine and a flexible torsion tube that is adapted to rotate an insulator housing or electrodes housed therein. The rotatable insulator housing can be used with either a bi-polar or mono-polar electrode configuration.

The present invention provides for further precision of the RF device by cycling a fluid solution passed the target surgical site. Fluid, such as a refrigerated saline solution, may be passed through a central fluid port in the RF device thereby providing suction or pressure to the target surgical site. Cycling a fluid passed the target surgical site allows for more precise control of thermal spread, in addition to removing air bubbles and debris from the immediate vicinity. Controlling thermal spread allows the surgeon to achieve the desired tissue effect at a precise target surgical site. Further, removal of air bubbles and debris from the target surgical site provides for better visualization and, therefore, more precise operation by the surgeon.

During surgery, it is typical to encounter both the need to cut tissue and coagulate bleeding tissue. Before the present invention, it took significant effort to change a tip of an RF device from a small cutting electrode to a large coagulating electrode. The surgeon was required to remove the first electrode (e.g., either the cutting or coagulating electrode) and place the second electrode at exactly the same spot in the surgical field that is viewed by the surgical camera. This is an extremely difficult maneuver because the surgical field viewed by the camera is usually very small.

To overcome this difficulty, surgeons typically use the same electrode to cut and coagulate tissue to avoid having to change from one electrode to another during surgery. For example, with reference to Table 1, a surgeon may switch from a coagulation/fulguration mode 1a to a cutting mode 1b so as to cut tissue with a coagulating electrode by bringing the electrode surface into contact with the tissue and increasing the energy flux dQ/dt. Alternatively, a surgeon may switch from a cutting/vaporization mode 2a to a desiccation mode 2b by coagulating with a cutting electrode by decreasing the energy flux dQ/dt. However, switching from a coagulation mode 1a to a cutting mode 1b, or from a cutting mode 2a to a desiccation mode 2b, with the same electrode of prior art RF devices requires the surgeon to contact the tissue with the electrode. This tissue contact results in eschar build-up on the electrode of the RF device and therefore increased electrical resistance at the energy transmitting surface and less precision of tissue effect. This results in numerous problems for the surgeon. Some problems are overcome by changing the duty cycle so as to reduce eschar build-up, but this is not always practical because of the change in performance. The eschar build-up, which reduces tip efficiency, may be temporarily overcome by increasing the voltage, but this is a temporary solution that quickly makes the eschar build-up even worse.

TABLE 1

| | | | Duty | | Tissue | Eschar |
|---|---|---|---|---|---|---|
| | Mode | Function | Cycle | dQ/dt | Contact | Build-up |
| la | Fulguration/ | Coagulate/Char | Low | Low | No | Minimum |
| | Coagulation | Tissue | | | | |
| lb | Cut | Divides/Char | Low | High | Yes | Maximum |
| | | Tissue | | | | |
| 2a | Cut/ | Divide Tissue | High | High | No | Minimum |
| | Vaporization | | | | | |
| 2b | Desiccation | Coagulate Tissue | High | Low | Yes | Maximum |

The present invention provides an electrosurgical RF device capable of switching from the cut mode to the coagulating mode without removing the device from the surgical camera field of view. In other words, with the present invention the surgeon is now able to have the correct electrode for the correct purpose without having to contact tissue. This results in minimal eschar build-up on the electrode and minimal eschar production in the patient. Accordingly, there are advantages to both the surgeon and the patient. The surgeon is able to use a single device capable of performing multiple electrosurgical modalities without the need to remove the RF device from the surgical site to change electrodes, and without the need to contact tissue, thereby quickening the surgical procedure, maintaining precision of tissue effect, and lengthening the life of the RF device. The patient benefits by having a reduced amount of eschar left in the body, thereby limiting the negative effects of eschar such as free radicals and their corresponding toxicity.

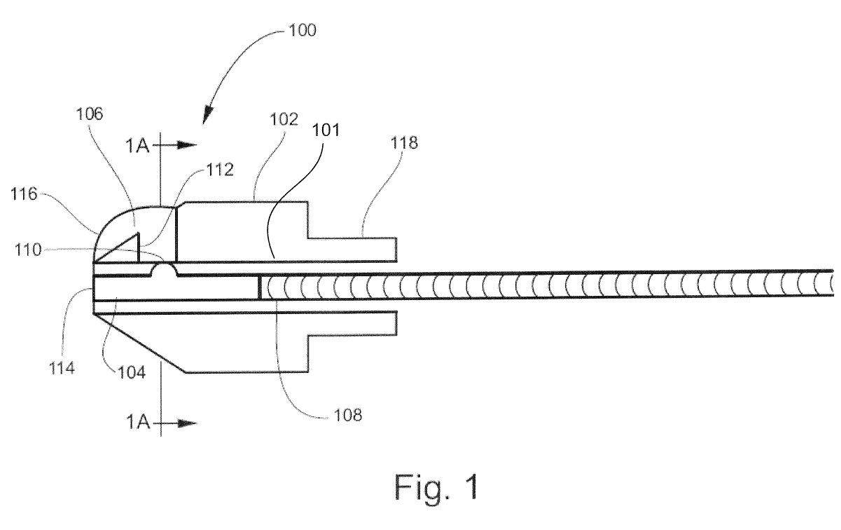

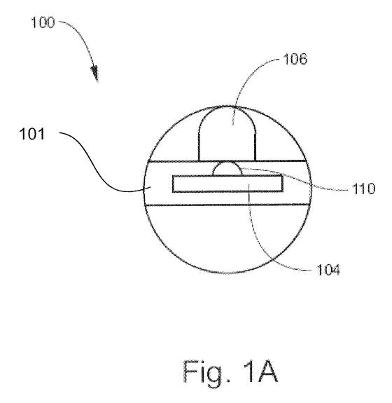

With reference to FIGS. 1-3, an RF device 100 according to an embodiment of the invention includes an insulator housing 102 to which a fixed coagulating electrode 106 is attached. A supply electrode 104 is disposed in a passage 101 within the housing 102 so that it can be moved between a first position in which the supply electrode 104 is withdrawn within the housing passage 101 as shown in FIG. 1 and a second position in which the supply electrode 104 extends distally outward from the housing passage 101. In the first configuration, the coagulating electrode 106 may be energized for a coagulation procedure and in the second configuration, the supply electrode 104 may be energized for a cutting procedure. The mobility of the supply electrode 104 allows the device 100 to quickly and easily change from one mode of operation to the other.

The RF device 100 will now be discussed in more detail. FIG. 1 shows a mono-polar RF device 100 in the coagulating mode. In this position, the insulator housing 102 is in electrical communication with the coagulating electrode 106, which may be a monolithic conductive element. The coagulating electrode 106 may have a rounded energy transmitting surface 116. For example, the energy transmitting surface may be a portion, such as a quarter, of a sphere or cylinder such that it has a large surface area. Some embodiments may actually have a plurality of coagulating electrodes 106 or may have a single coagulating electrode with multiple energy transmitting surfaces 116.

A supply electrode 104 is disposed within the insulator housing 102 and is slidable therein. Supply electrode 104 may have a flat planar surface with a sharp distal edge, and may further contain a bend therein to aid in bringing the RF device closer to the target surgical site. An electrical contacting surface 110 on the supply electrode 104 causes the supply electrode 104 and the coagulating electrode 106 to be electrically conductive. The supply electrode 104 is, in turn, directly or indirectly connected to an RF generator (not shown) that delivers energy to the RF device 100. Electrical contacting surface 110 may be integral to, or separate from, the supply electrode 104. A flexible actuator member 108 is connected to supply electrode 104 and can retract or extend the supply electrode 104 between a proximal position and a distal position. In the proximal position, the electrical contacting surface 110 is in electrical contact with the coagulating electrode 106.

In a preferred embodiment, a coagulating waveform from the RF generator is delivered through the supply electrode 104, which is in a proximal position, to the coagulating electrode 106. The coagulating waveform may be selected via an activating footswitch or some other mode-switching device (not shown). A coagulating waveform preferably delivers a low energy flux dQ/dt in a manner that will not cause eschar build-up on the coagulating electrode 106, but that allows for effective tissue coagulation without the need to contact tissue with the coagulating electrode 106. Further, the material of the supply electrode 104 and the coagulating electrode 106 may be any suitable conductive material and capable of delivering energy to a target site. Preferably, the electrode material is highly conductive, resistant to oxidation, and may be coated with a Teflon® or an elastomeric silicone coating. Specific material types of the electrode(s) for this and other embodiments of the invention may include brass, carbon-amorphous, graphite, copper, copper graphite, copper tellurium, copper tungsten, copper zirconium diboride, gold or gold alloy, electrographite, metal graphite, molybdenum, palladium or palladium alloys, platinum or platinum alloys, plated base metal, resin bonded graphite, silver or silver alloys, silver copper, silver cadmium oxide, silver graphite, silver molybdenum, silver nickel, silver tin oxide, silver tungsten, silver tungsten carbide, tungsten, and tungsten carbide.

FIG. 1A is a cross-sectional view taken along the line 1A-1A in FIG. 1. The coagulating electrode 106 is shown having a large surface area for spreading energy flux to tissue, and supply electrode 104 is shown having a small surface area for concentrating energy flux to tissue. The energy transmitting surface of the coagulating electrode 106 is preferably larger than that of the supply electrode 104. Further, the supply electrode 104 is shown having a wide and planar shape, but the width, shape, or angle of the supply electrode 104 may vary, and the supply electrode may take on other shapes such as a pin or loop, or have a bend formed therein, for example. In a typical embodiment, the coagulating electrode may have an energy transmitting surface area of at least 0.01 square inches, while the cutting electrode has an energy transmitting surface of no more than 0.002 square inches.

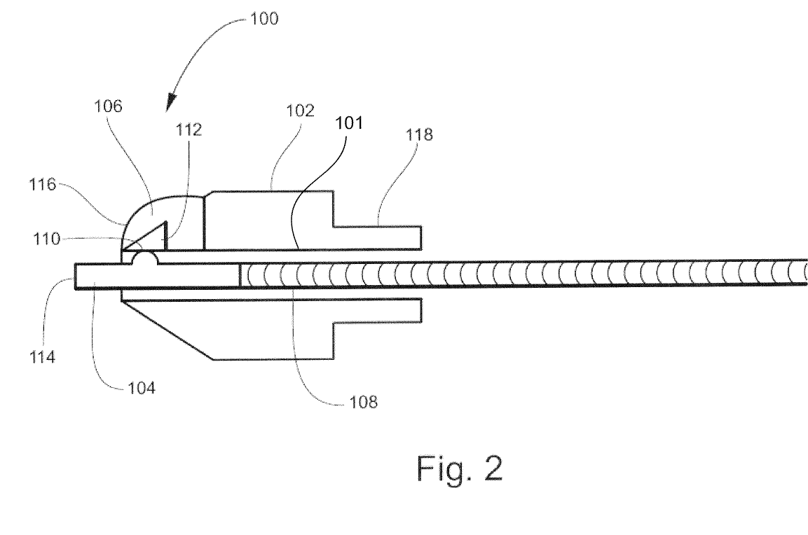

FIG. 2 shows the mono-polar RF device 100 in a cutting mode. To get to the cutting mode, a flexible actuator member 108 may push or drive the supply electrode 104 distally until the electrical contact surface 110 is disconnected from the coagulating electrode 106 by residing in, or being exposed to, a void 112 that may be formed in the coagulating electrode 106. This puts the supply electrode 104 in a distal position such that the distal end thereof is exposed so that it can cut tissue. The energy transmitting surface 114 is preferably at a distal end of supply electrode 104, but may include more than a distal surface of the supply electrode 104. The device 100 may be returned to coagulating mode by causing the flexible actuator member 108 to withdraw the supply electrode 104 back to the position shown in FIG. 1.

It will be understood by those of ordinary skill in the art that the flexible actuator member 108 may be operatively connected to any mechanism suitable for causing selective translation of the flexible actuator member relative to the insulator housing 102. As discussed in more detail below, the insulator housing 102 will typically be mounted to a flexible support member through which the flexible actuator member 108 may be slidably disposed.

In some embodiments, the supply electrode 104 may be energized by placing an activating footswitch or some other mode-switching device in the “cut” position. This delivers a high energy flux dQ/dt in a manner that will not cause eschar build-up on the supply electrode 104, but that allows for effective tissue cutting without the need to contact tissue with the supply electrode 104. In both the cutting and coagulating mono-polar modes, a return electrode may be positioned in contact with the patient at a location removed from the surgical site.

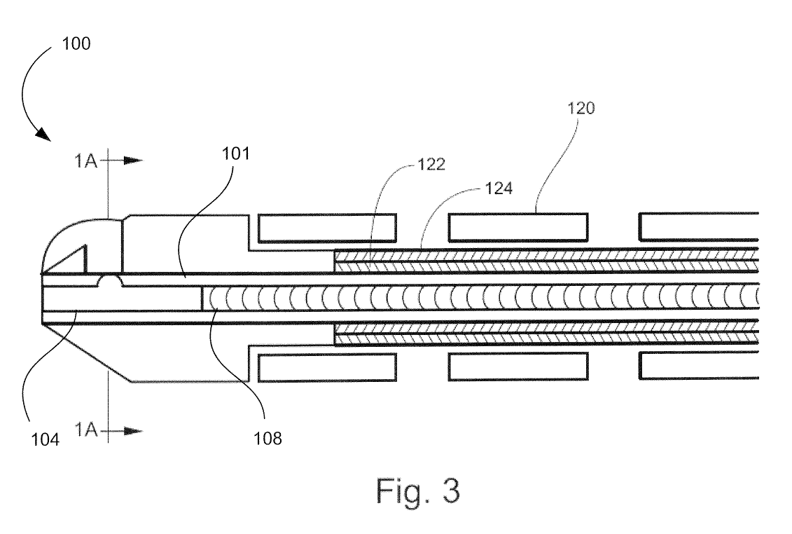

As shown in FIG. 3, the supply electrode 104 may be slidably disposed within a torsion-transmitting flexible tube 122 and the insulator housing 102. The flexible tube 122 is capable of rotating the insulator housing 102 so that the coagulating electrode 106 and supply electrode 104 may be presented to a surgical site in various angular orientations. The flexible tube 122 is sealed by an insulating media 104, which may be, but is not limited to, a Pebax® heat shrink material.

In one embodiment of the device, a flexible spine or other support member 120 is positioned around a recessed portion 118 of the insulator housing 102. The flexible spine 120 may be made up of vertebrae that may be formed as a single molded part or may be formed as a plurality of vertebrae individually formed and strung together. The flexible spine 120 allows the RF device to bend in an up-down and/or a left-right direction such that the coagulating electrode 106 and supply electrode 104 can take on even further angular and positional orientations. Flexible support members of this type are described in detail in U.S. application Ser. No. 11/643,740, filed Dec. 20, 2006, U.S. application Ser. No. 12/119,799, filed May 13, 2008, and U.S. application Ser. No. 12/399,471, filed Mar. 6, 2009, the complete disclosures of which are incorporated herein by reference in their entirety. Preferably, a proximal end of the insulator housing 102 is connected to a flexible support member 120, and a proximal end of the flexible support member 120 may be connected to a rigid or semi-rigid tubular member (not shown), which in turn may be connected to a handle (not shown). However, the RF device 100 of the present invention may be used without a flexible support member 120 as well.

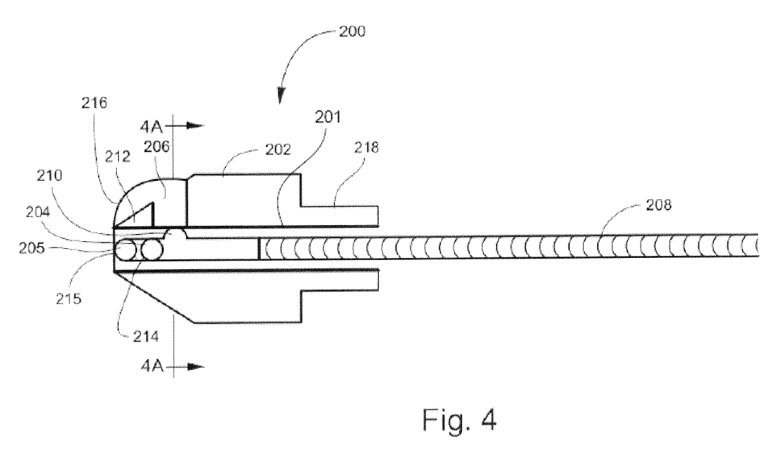

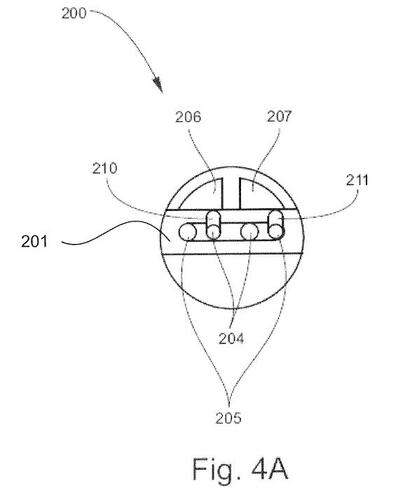

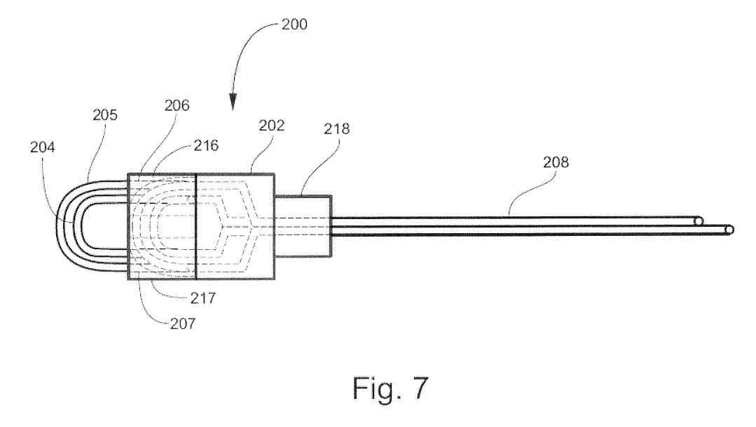

FIGS. 4-7 show a bi-polar RF device that is similar in function to the mono-polar RF device, but a return electrode is included near the supply electrode of bi-polar RF device 200. For example, FIG. 7 shows a top view of the RF device 200 in the bi-polar cutting mode. In the bi-polar cutting mode, a supply electrode 204 may be near a return electrode 205, both of which may be formed as wire loops. Both the supply electrode 204 and a return electrode 205 may be actuatable or movable within the housing passage 201 from a proximal position to a distal position, as shown in FIG. 7 with the proximal position shown in phantom. In the embodiment shown in FIG. 7, the return electrode 205 is the outer/larger loop and the supply electrode 204 is the inner/smaller loop, but this may be reversed. These loops may be proximally retracted together by a flexible actuator member 208 to allow the RF device 200 to function in the coagulating mode, or they may be distally extended to function in the cutting mode. Two coagulating electrodes 206, 207 are also depicted in FIG. 7 with phantom lines for the sake of clarity.

FIG. 4 shows the RF device 200 in a coagulating mode. The cross-sectional view in FIG. 4 shows return the electrode 205 and the supply electrode 204. Also shown is the coagulating electrode 206 that works in conjunction with supply electrode 204 via electrical contacting surface 210. Not shown in FIG. 4 (because it is in the left hand side cross-section when looking distally) is the other coagulating electrode 207 that works in conjunction with return electrode 205 via electrical contacting surface 211. Both coagulating electrodes 206, 207 are shown in the front cross-sectional view, depicted in FIG. 4A. As can be seen, the first coagulating electrode 206 is in electrical communication with the supply electrode 204 (inner loop) via an electrical contacting surface 210, and the second coagulating electrode 207 is in electrical communication with the return electrode 205 (outer loop) via an electrical contacting surface 211. The return electrode 205, supply electrode 204, and corresponding coagulating electrodes 206, 207 can take on different shapes, but in the bi-polar mode a return electrode is proximate, or adjacent, to the supply electrode.

To function in the coagulating mode as shown in FIG. 4, a flexible actuator member 208 retracts the supply electrode 204 and return electrode 205 until the electrical contacting surfaces 210, 211 (211 not shown in FIG. 4) are housed within, or facially disposed in, voids 212, 213 (213 not shown in FIG. 4). The actuator 208 may electrically connect, either directly or indirectly, the supply electrode 111 and return electrode 112 to the RF generator. Alternatively, wires (not shown) may extend to the supply electrodes 204, 206 to electrically connect the supply electrodes 204, 206 to the RF generator. In some embodiments, the coagulating waveform from the RF generator may be selected via an activating footswitch (not shown) or some other mode-switching device. The coagulating waveform delivers the low energy flux dQ/dt in a manner that does not require tissue-electrode contact and that will not cause eschar build-up on the coagulating electrodes 206, 207. The energy flux may travel through energy transmitting surfaces 216, 217 of the supply coagulating electrode 204 and the return coagulating electrode 205. These energy transmitting surfaces 216, 217 may be in the shape of a portion of a sphere or cylinder such that it has a large or bulbous surface area. Further, the material of the supply coagulating electrode 204 and the return coagulating electrode 205 may be any suitable conductive material and capable of delivering energy to a target site. Preferably, the electrode material is highly conductive, resistant to oxidation, and may be coated with a Teflon® or an elastomeric silicone coating. Specific material types of the electrode(s) for this and other embodiments of the invention may include brass, carbon-amorphous, graphite, copper, copper graphite, copper tellurium, copper tungsten, copper zirconium diboride, gold or gold alloy, electrographite, metal graphite, molybdenum, palladium or palladium alloys, platinum or platinum alloys, plated base metal, resin bonded graphite, silver or silver alloys, silver copper, silver cadmium oxide, silver graphite, silver molybdenum, silver nickel, silver tin oxide, silver tungsten, silver tungsten carbide, tungsten, and tungsten carbide.

Affixed to the insulator housing 202 are the coagulating electrodes 206, 207. The coagulating electrodes 206, 207 may contain voids 212 that are capable of housing or facially opposing the electrical contacting surfaces 210, 211 of the looped supply electrode 204 and the looped return electrode 205, respectively, so as to disengage electrical connection between the coagulating electrodes 206, 207 and an electrical waveform (RF) generator (not shown). Coagulating electrodes 206, 207 are also capable of engaging the electrical contacting surfaces 210, 211 when the RF device 200 is to function in the coagulating mode.

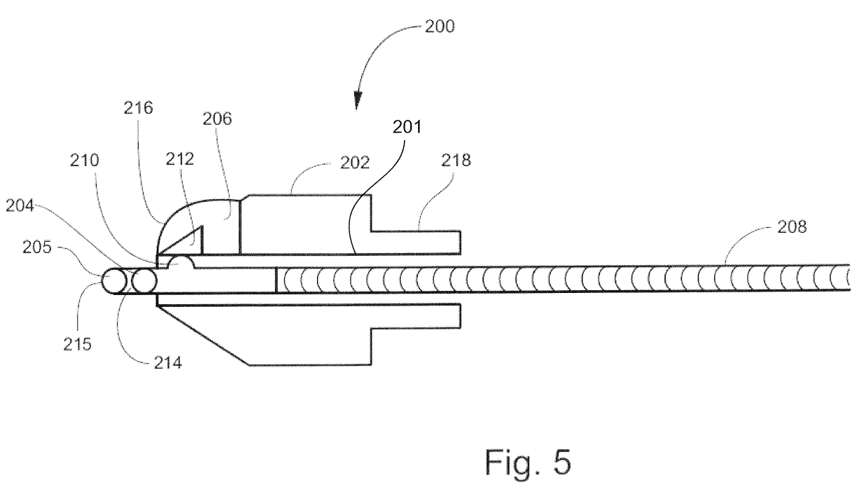

In the cutting mode, as shown in FIG. 5, the flexible actuator member 208 may push or drive the preferably looped supply electrode 204 and return electrode 205 distally until the electrical contacting surfaces 210, 211 are disconnected from the coagulating electrodes 206, 207. This puts the supply electrode 204 and the return electrode 205 in an exposed position to cut tissue. In one preferred embodiment, the supply electrode 204 is energized by placing an activating footswitch (not shown) or some other mode-switching device in the “cut” position. This delivers the high energy flux dQ/dt described above in a manner that does not require tissue-electrode contact and that will not cause eschar build-up on the supply electrode 204 or the return electrode 205. The energy flux may travel through energy transmitting surfaces 214, 215 of the preferably looped supply electrode 204 and return electrode 205, respectively. The device 200 may be returned to coagulating mode by causing the actuator 208 to withdraw the looped supply electrode 204 and return electrode 205 back to the position shown in FIG. 4.

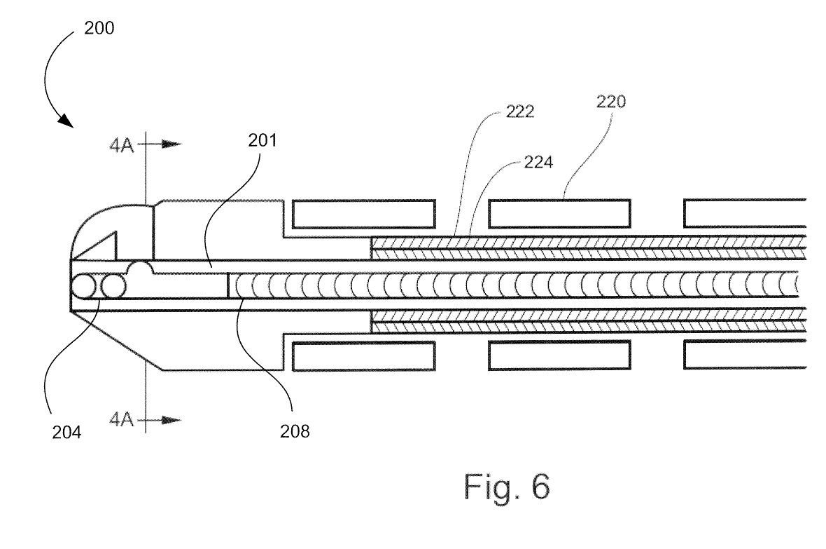

As shown in FIG. 6, the supply electrode 204 and the return electrode 205 may be slidable within a torsion-transmitting flexible tube 222 and an insulator housing 202. The flexible tube 222 is capable of rotating the insulator housing 202 so that the coagulating electrodes 206, 207 and supply electrodes 204, 205 may be presented to a surgical site in various angular orientations. The flexible tube 222 may be sealed by an insulating media 224, which may be, but is not limited to, a Pebax® heat shrink material. In one preferred embodiment of the device, a flexible spine or support member 220 is positioned on a recessed portion 218 of the insulator housing 202. The flexible spine 220 may be made up of vertebrae that may be formed as a single molded part or may be formed as a plurality of vertebrae individually formed and strung together. The flexible spine 220 allows the RF device to bend in an up-down and/or a left-right direction such that the coagulating electrodes 206, 207 and supply electrodes 204, 205 can take on even further angular and positional orientations. Preferably, a proximal end of the insulator housing 202 is connected to a flexible support member 220, and a proximal end of the flexible support member 220 may be connected to a rigid or semi-rigid tubular member (not shown), which in turn may be connected to a handle (not shown). However, the RF device 200 of the present invention may be used without a flexible support member 120 as well.

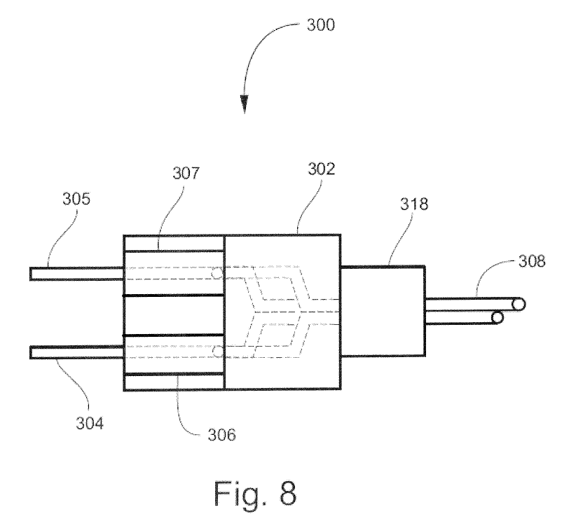

FIG. 8 shows another embodiment where a supply electrode 304 and a return electrode 305 are configured as protruding pins. Other configurations for the return and supply electrodes are also possible. Coagulating electrodes 306, 307 may have a large or bulbous surface for delivering energy flux to tissue in a suitable coagulating manner. Electrical contacting surfaces 310, 311 may be attached to the supply and return electrodes 304, 305, respectively.

The loop configuration depicted in FIG. 7 may be used for skin surfaces where the tissue has been removed. The parallel protruding pin configuration of FIG. 8, on the other hand, may act as a scalpel as the tissue between the return electrode 305 and the supply electrode 304 is cut. In some embodiments, the position of the supply electrode and the return electrode may be reversed.

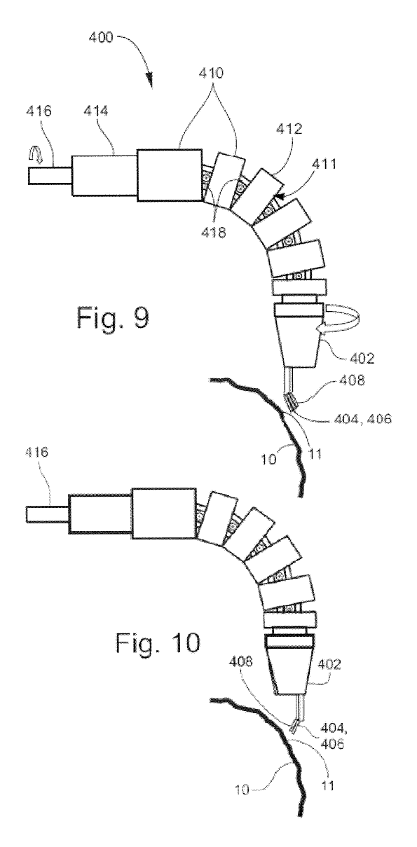

With reference now to FIGS. 9-12, an RF device 400 according to another embodiment of the invention includes a rotatable insulator housing 402 to present a variety of RF electrode orientations relative to the target tissue 10. FIGS. 9-12 illustrate a bi-polar RF device 400, but the features described herein may also be applied to mono-polar devices as well. For example, instead of having multiple electrodes, the insulator housing 402 may contain a single electrode that functions as a supply electrode. In such a case, a return electrode would be positioned away from the supply electrode, such as under the patient. Accordingly, the RF device 400 contains at least one electrode with a plurality of energy transmitting surfaces.

Additionally, the devices in FIGS. 9-14 may utilize the slidable mechanism described above with respect to FIGS. 1-8. As such, a flexible actuator member 416 or another member may extend distally or retract proximally the at least one electrode on the RF devices 400, 500. Electrical conductivity may be accomplished through the flexible actuator member 416, 516 or through wires. Use of an actuator may prevent complications of twisting wires near the insulator housing 402, 502.

FIG. 9 illustrates a bi-polar rotatable RF device 400 in a coagulation mode. The RF device 400 may comprises a flexible spine or other support member 410 and a rotating, cannulated flexible actuator member 416 adapted to rotate an insulator housing 402 about a central axis thereof in a manner that positions the faces of electrodes 404, 406 to be generally tangential to the target tissue 10, thus forming a tangential electrode configuration. The flexible spine 410 may be made up of vertebrae 412 that may be formed as a single molded part or may be formed as a plurality of vertebrae individually formed and strung together. The flexible spine 410 defines a passage 411 through which the flexible actuator member 416 is passed. The flexibility of the spine 410 allows the RF device 400 to bend in an up-down and/or a left-right direction such that the electrodes 404, 406 (and optionally 408) can take on even further angular and positional orientations. Preferably, a proximal end of the insulator housing 402 is connected to flexible support member 410, and a proximal end of the flexible support member 410, such as recessed portion 414, may be connected to a rigid or semi-rigid tubular member (not shown), which in turn may be connected to a handle (not shown). However, the RF device 200 of the present invention may be used without a flexible support member 410 as well.

It will be understood that the axis of rotation of the insulator housing 402 is established by the configuration of the flexible support member 410. Specifically, the axis of rotation will be established by the orientation of the distal end of the support member 410. If the support member 410 is in a curved orientation, the axis of rotation will be a tangent to the curve through the center line of the support member at its distal end.

The relatively large surface area of the electrodes 404, 406 broadens the flow of energy flux into the tissue thereby allowing coagulation to occur. In a typical embodiment, the total energy transmitting surface area of the coagulating electrodes is at least 0.01 square inches. Electrodes 404, 406, and 408 are depicted as having a flat planar surface with a sharp distal edge, and may further contain a bend therein to aid in bringing the RF device closer to the target surgical site. Preferably, in the coagulation mode electrode 406 is at the supply potential, electrode 404 is at the return potential, and electrode 408 is disabled (i.e., isolated). Supply and return wires 418 can be routed around the circumference of the flexible actuator member 416 back to an RF generator or ground in a manner that allows 360 degree rotation of the rotatable tip 400. Alternatively, as discussed above, an actuator may electrically connect the electrodes to an RF generator or ground. The tangential electrode configuration shown in FIG. 9, which allows for effective coagulation without the need for tissue-electrode contact, may be initiated by the surgeon selecting the “coagulate” pedal of a footswitch (not shown) that is indirectly connected to the RF device 400. In a mono-polar device, both electrodes 404, 406 may be at the supply potential and the return path is through an electrode that may be positioned beneath the patient. Alternatively, there may be a single supply electrode on the RF device 400. In either case, the supply current waveform may be pre-programmed in the generator to coagulate bleeding tissue sites, such as tissue site 11, by heating but without any significant cell damage.

Preferably, the electrode material of the rotatable RF devices is highly conductive, resistant to oxidation, and may be coated with a Teflon® or an elastomeric silicone coating. Specific material types of the electrode(s) for this and other embodiments of the invention may include brass, carbon-amorphous, graphite, copper, copper graphite, copper tellurium, copper tungsten, copper zirconium diboride, gold or gold alloy, electrographite, metal graphite, molybdenum, palladium or palladium alloys, platinum or platinum alloys, plated base metal, resin bonded graphite, silver or silver alloys, silver copper, silver cadmium oxide, silver graphite, silver molybdenum, silver nickel, silver tin oxide, silver tungsten, silver tungsten carbide, tungsten, and tungsten carbide.

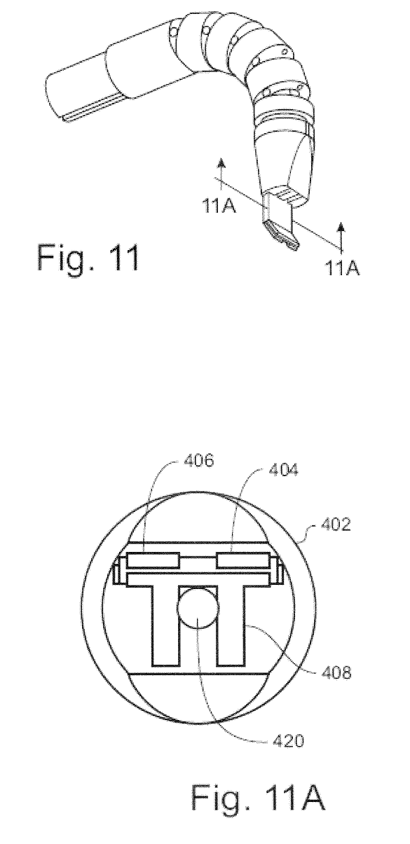

FIG. 10 illustrates the bi-polar rotatable RF device 400 in a cutting mode. The rotatable RF device 400 is rotated approximately 180 degrees from the coagulation mode to present the preferably flat distal edge of electrodes 404, 406, 408 in an orientation roughly perpendicular to the target tissue 10. When the one or more electrodes are in a perpendicular orientation to the target tissue 10, the sharp (small surface area) edge of the electrode(s) presents a very high energy flux to the tissue and ablates or cuts the cells nearby; i.e., sufficient energy flux is delivered to the tissue to vaporize the cells and thereby cut through the tissue. For example, as shown in the cross-sectional view in FIG. 11A, electrodes 404 and 406 have a small surface area at their edge, and electrode 408 is shown to have a double-legged “T” or a Π configuration. While the electrodes in FIGS. 9-12 are shown to have a flat, large area on one surface and a square, small area on another surface, these surfaces may take on other configurations such as a looped, circular, round, or other shaped structure. Preferably, however, one surface of the electrode(s) will have a small surface area and another surface of the electrode(s) will have a larger surface area. The small surface area presented in the cutting mode will typically be less than or equal to 0.002 square inches.

In the bi-polar example of FIGS. 9-11A, when an RF generator (not shown) is set to the “cut” mode, electrode 408 preferably functions as the supply electrode and its potential may be generated by a preset software algorithm in the RF generator, and electrodes 404, 406 preferably function as the return electrodes both set to ground potential. Thus, the resulting current path is from the supply electrode 408 across the gap and then to either of the two return electrodes 404, 406. As referred to above, this cutting mode and the corresponding potential of each of the electrodes may be selected by the surgeon depressing the “cut” pedal of the footswitch connected to the RF generator. In conjunction with setting the RF generator to the “cut” mode, the rotatable RF device 400 is preferably rotated to the perpendicular orientation for effective cutting without the need for tissue-electrode contact. A mono-polar configuration is not shown, but would function similarly and the current path would flow from the supply electrode through the patient to a return electrode positioned beneath the patient.

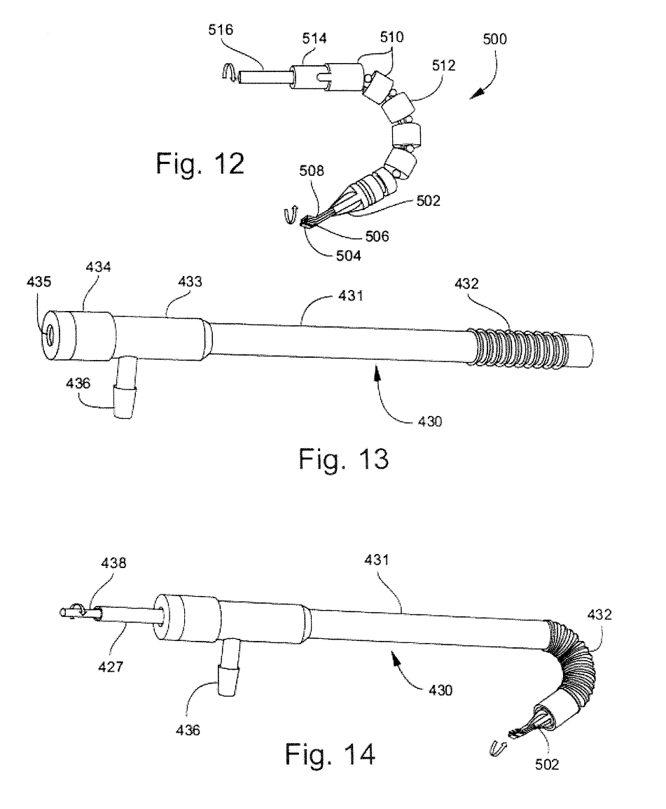

As shown in FIG. 12, the bi-polar or mono-polar rotatable RF device 500 may also be modified to have a torsion drive cable 516 as the flexible actuator member instead of a cannulated core passageway. The torsion drive cable 516 may be hollow but is preferably solid or filled with multiple cables to increase torsional resistance. The torsion drive cable 516 is preferably connected to the insulator housing 502 and upon rotation of the torsion drive cable 516 the insulator housing 502 is rotated. An annular gap between the inner diameter of the flexible spine or support member 510 (or inner diameter of recessed portion 514) and the outer diameter of the torsion drive cable 516 provides space for the supply and return wires (not shown), which may extend from the electrodes 504, 506, 508 through the flexible spine 510 to an RF generator (not shown), similar to that described for FIG. 9. Alternatively, an actuator may be used, as described above, to electrically connect the electrodes to the RF generator (and optionally slidably actuate the electrodes). The flexible spine 510 also has a recessed portion 514 on which an outer tube (such as tube 437 in FIG. 14) of the RF device 500 may sit.

It is advantageous to control the temperature of the tissue that is exposed to RF energy to temperatures below 60 degrees Centigrade. It is also apparent that air bubbles which are formed adjacent to the application site of high flux RF energy have a negative effect on the surgeon's visibility of the electrodes. To overcome these limitations in the prior art, a refrigerated or unheated saline solution may be flowed across the application site of the RF energy. In one preferred embodiment, the saline solution may be drawn out of the application site, such as an articular capsule, by vacuum or reverse pumping, thereby carrying bubbles away and drawing refrigerated or unheated saline solution across the RF site. In a second embodiment, the saline solution may be pushed into the capsule or across the RF site by pumping or gravity, thereby feeding fresh saline across the RF site and carrying bubbles away to a corresponding vent appliance (e.g., cannula, shavers, natural leakage pathways in the capsule, etc.) (not shown).

Some embodiments of the invention may include a fluid passage for passing a cooling fluid to the operating end of the RF device. FIG. 11A show a central fluid passage in the insulator housing 402 that terminates at its distal end in a port 420. This passage and port 420 may be incorporated into the bi-polar or mono-polar rotatable RF tip 400 for cycling a fluid solution passed the target RF site 11. The central port 420 connects to a central, co-axial core comprising the flexible actuator member or torsion tube 416 and a tube (not shown) that interconnects at a handle (not shown) which is in fluid communication with a supply source or return reservoir (not shown). The central port 420 may be slightly smaller in diameter than the coaxial core to prevent entrance of debris that is large enough to plug the pathway of the coaxial core. The central port 420 cycles fluid, that is preferably refrigerated, passed target RF site 11 to thereby control the temperature of the tissue exposed to RF energy and to remove air bubbles and debris from the vicinity of the target RF site 11.

An alternative embodiment of the invention employs a circumferential fluid pathway rather than a central fluid pathway. FIG. 13 shows a sheath 430 comprising a semi-rigid tube 431 and a bendable portion 432 near the distal end of sheath 430 for housing, for example, the flexible spine 510 of RF device 500. The semi-rigid tube 431 is preferably capable of withstanding −2.0 psig suction pressure and the bendable portion 432 may be formed in an accordion-like fashion. The sheath 430 is fixed to a molded hub 433 with a tube port 436 for transferring fluid to or from the sheath 430. The hub may be capped with a silicone seal 434 which seals around a device, such as RF device 400 or 500, that is inserted through main port 435 of hub 433.

FIG. 14 shows the RF device 500 of FIG. 12 inserted into the hub 433 and sheath 430 of FIG. 13. A fluid pathway is formed between the inner diameter of the sheath 430 and the outer diameter of the inserted device, such as outer diameter of outer tube 537 of RF device 500. The fluid pathway is in fluid communication with the hub port 436 to allow for fluid delivery to, or removal from, the target site 401. The bendable portion 432 of the sheath 430 conforms to the bendable shape of the flexible spine 510. Protruding from the distal end of the sheath 430 is the insulator housing 502. Protruding from the proximal end is the outer tube 537 and inner tube 538 of the inserted device (such as RF device 500). Inner tube 538 may be the flexible actuator member 516 (torsion drive cable) or the cannulated flexible actuator member 416 depending on which RF device is inserted. Preferably, there is a rotation means in the handle to cause the inner tube 538 to rotate which, in turn, rotates the insulator housing 502. Additionally, fluid transport tubing that extends from the hub port 436 to the handle may pass through a finger-actuated valve in the handle to turn fluid flow on or off.

The foregoing description of the invention is merely illustrative, and it is understood that variations and modifications can be effected without departing from the scope or spirit of the invention as set forth in the following claims.

What is claimed is:

1. A radio frequency (RF) device for use in electrosurgery to deliver electrical current from an electrical source to target tissue of a patient, the RF device comprising:
a flexible support member having an actuator passage extending therethrough from a proximal support member end to a distal support member end;
a flexible actuator member at least partially disposed within the actuator passage, the flexible actuator member being selectively rotatable relative to the flexible support member and having a distal actuator end adjacent the distal support member end;
an insulator housing having a proximal side and a distal side, the insulator housing being connected on its proximal side to the distal actuator end so that when the flexible actuator member is rotated, the insulator housing rotates about a rotation axis through the distal actuator end, the insulator housing being rotatable between at least first and second angular orientations; and
at least one electrode in selective communication with the electrical source, the at least one electrode extending distally from the distal side of the insulator housing and having first and second energy transmitting surfaces configured so that if the first energy transmitting surface is positioned to deliver electrical current to the target tissue with the insulator housing in the first angular orientation, rotation of the insulator housing to the second angular orientation positions the second energy transmitting surface for transfer of electrical current to the target tissue.

2. The RF device of claim 1, wherein the first energy transmitting surface is configured for delivering electrical current for coagulation of at least a portion of the target tissue and the second energy transmitting surface is configured for delivering electrical current to cut at least a portion of the target tissue.

3. The RF device of claim 1, wherein the first energy transmitting surface has a tissue engaging area of at least 0.01 square inches and the second energy transmitting surface has a tissue engaging area of no more than 0.002 square inches.

4. The RF device of claim 1, wherein the at least one electrode comprises a coagulating electrode having a first coagulating electrode portion attached to the insulator housing and extending distally from the insulator housing parallel to the rotation axis and a second coagulating electrode portion extending distally from a distal end of the first coagulating electrode portion and terminating in a coagulating electrode tip, the second coagulating electrode portion being angled relative to the first coagulating electrode portion, wherein a first surface of the second coagulating electrode portion comprises at least a portion of the first energy transmitting surface.

5. The RF device of claim 4, wherein the at least one electrode comprises a cutting electrode extending distally from the insulator housing and terminating in a cutting electrode tip adjacent but spaced apart from the coagulating electrode tip, the cutting electrode tip having a tip surface area comprising at least a portion of the second energy transmitting surface.

6. The RF device of claim 1, further comprising a fluid passage terminating in a port in the distal side of the insulator housing, the fluid passage and the port being positioned and configured for delivering fluid to or removing fluid from the target site in both the first and second angular orientations.

7. The RF device of claim 1, wherein the first angular orientation differs from the second angular orientation by about 180 degrees.

8. The RF device of claim 1, wherein the flexible support member comprises a plurality of vertebrae connected to one another by integrally formed web members.

9. The RF device of claim 1, wherein the distal support member end is attached to the proximal side of the insulator housing so that the distal support member end rotates about the rotation axis with the insulator housing.

10. A radio frequency (RF) device for use in electrosurgery to deliver electrical current from an electrical source to target tissue of a patient, the RF device comprising:
an insulator housing having a housing passage extending therethrough, the housing passage having a proximal passage opening in a proximal side of the insulator housing and a distal passage opening in a distal side of the insulator housing;
a first electrode attached to the distal side of the insulator housing, the first electrode having an external first energy transmitting surface and an internal electrical contact surface exposed to the housing passage;
a second electrode in selective communication with an electrical energy source, the second electrode having a second energy transmission surface at a distal electrode end and an electrical supply contact and being slidably disposed within the housing passage so that it is movable between a first position in which the second electrode is withdrawn within the housing passage and the electrical supply contact is in electrical communication with the internal electrical contact of the first electrode and a second position in which at least a portion of the second electrode extends distally outward from the distal passage opening and the electrical supply contact is not in electrical communication with the internal electrical contact of the first electrode; and
a flexible actuator member having a distal actuator end slidably disposed within the housing passage, the distal actuator end being attached to a proximal end of the second electrode so that axial translation of the flexible actuator member causes corresponding motion of the second electrode relative to the housing passage.

11. The RF device of claim 10, further comprising:
a flexible support member having an actuator passage extending therethrough from a proximal support member end to a distal support member end,
wherein the proximal side of the insulator housing is attached to the distal support member end and a proximal portion of the flexible actuator member is slidably disposed within the actuator passage.

12. The RF device of claim 11, wherein the flexible support member comprises a plurality of vertebrae connected to one another by integrally formed web members.

13. The RF device of claim 11, wherein the insulator housing is selectively rotatable about an axis through the distal support member end.

14. The RF device of claim 10, wherein the first electrode comprises a monolithic body having a void in communication with the housing passage adjacent the internal electrical contact surface, the void being configured and positioned so that when the second electrode is in the second position, the electrical supply contact is exposed to the void.

15. The RF device of claim 10 wherein the first energy transmitting surface is a curved surface.

16. The RF device of claim 10, wherein the second energy transmitting surface is substantially smaller than the first energy transmitting surface.

17. The RF device of claim 10, wherein the first energy transmitting surface has a tissue engaging area of at least 0.01 square inches and the second energy transmitting surface has a tissue engaging area of no more than 0.002 square inches.

18. The RF device of claim 10, wherein the second electrode comprises at least one wire loop.

19. The RF device of claim 10, wherein at least one of the first and second electrodes is configured for delivering electrical current for coagulation of at least a portion of the target tissue.

20. The RF device of claim 10, wherein at least one of the first and second electrodes is configured for delivering electrical current to cut at least a portion of the target tissue.
